(19) United States
(12) Patent Application Publication
(10) Pub. No.: US 2021/0275700 A1
(43) Pub. Date: Sep. 9, 2021

(54) UV DISINFECTION PACKAGE

(71) Applicant: Shenzhen COOGHI Funkids Technology Co., Ltd, Shenzhen (CN)

(72) Inventor: JUN ZHANG, Shenzhen (CN)

(21) Appl. No.: 15/931,622

(22) Filed: May 14, 2020

(30) Foreign Application Priority Data
Mar. 6, 2020 (CN) 202020272319.0

(51) Int. Cl. A61L 2/10

(52) U.S. Cl. CPC A61L 2/10; A61L 2202/182; A61L 2202/11; A61L 2202/23; A61L 2202/122; A61L 2202/123; A61L 2202/16; A61L 2202/121

(57) ABSTRACT

A UV disinfection package includes a main body; a bracket fixedly installed in the main body, wherein the bracket includes a light-transmitting hole, a storage layer and a foot, the foot being fixedly connected to the storage layer, and the light-transmitting hole being provided on the storage layer; a UVC lamp group fixedly installed on the main body; a reflector fixedly connected to the UVC lamp group, and an aluminum film covering the inner wall of the main body.

### CROSS REFERENCE TO RELATED APPLICATION

This application claims priority to Chinese Patent Application No. 202020272319.0, filed on Mar. 6, 2020, which is hereby incorporated by reference herein as if set forth in its entirety.

### BACKGROUND

### 1. Technical Field

The present disclosure generally relates to technical field of disinfection appliances, and particularly to a UV disinfection package.

### 2. Description of Related Art

UV disinfection package plays a very good role as a sterilization and disinfection product. In people's daily life, many things around us contain a lot of bacteria and viruses. For example, there are many invisible bacteria or viruses directly affecting our health on the surface of outdoor air dried underwear, mobile phones, wallets, keys and so on. Especially in the period of novel coronavirus epidemic, the sterilization and disinfection of daily articles are particularly important. But in daily life, people often rely on disinfection water and alcohol for disinfection, which is easy to cause certain harm to the human body, and the disinfection to daily articles is not comprehensive or thorough enough.

### BRIEF DESCRIPTION OF THE DRAWINGS

Many aspects of the present embodiments can be better understood with reference to the following drawings. The components in the drawings are not necessarily drawn to scale, the emphasis instead being placed upon clearly illustrating the principles of the present embodiments. Moreover, in the drawings, all the views are schematic, and like reference numerals designate corresponding parts throughout the several views.

FIG. 1 is an isometric view of a UV disinfection package according to an embodiment.

FIG. 2 is an exploded isometric view of a UV disinfection package according to an embodiment.

FIG. 3 is another isometric view of a UV disinfection package according to an embodiment.

FIG. 4 is a schematic diagram of a UVC lamp group of a UV disinfection package according to an embodiment.

FIG. 5 is a schematic diagram of a IN disinfection package when it is open according to an embodiment.

FIG. 6 is a schematic diagram of a bracket of a UV disinfection package according to an embodiment.

### DETAILED DESCRIPTION

The disclosure is illustrated by way of example and not by way of limitation in the figures of the accompanying drawings, in which like reference numerals indicate similar elements. It should be noted that references to “an” or “one” embodiment in this disclosure are not necessarily to the same embodiment, and such references can mean “at least one” embodiment.

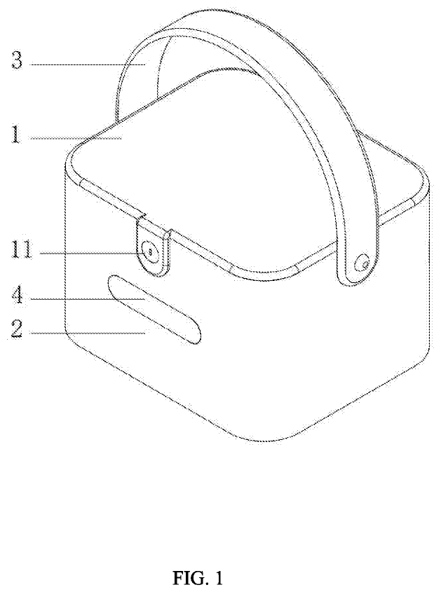

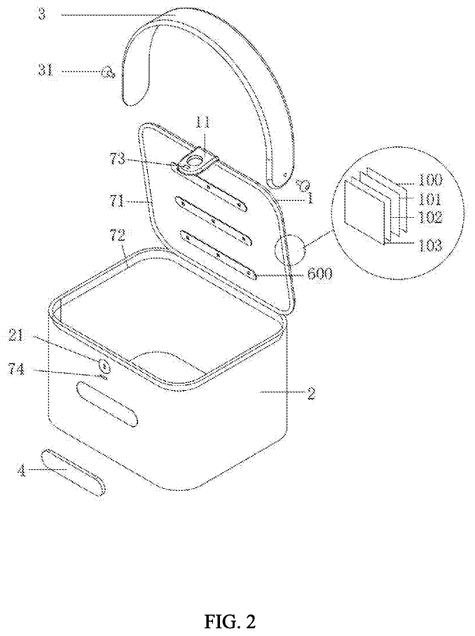

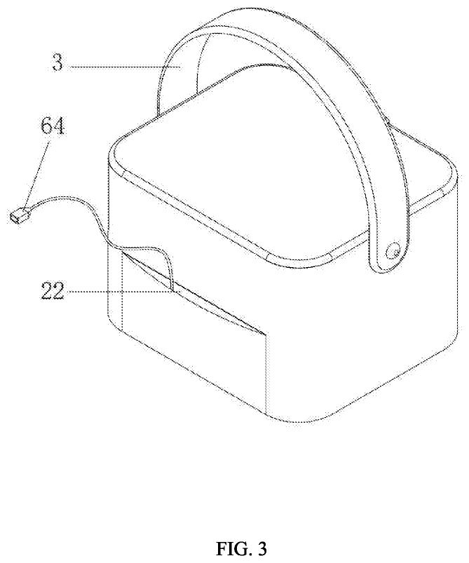

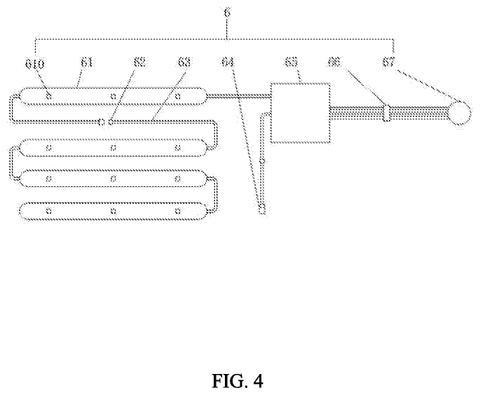

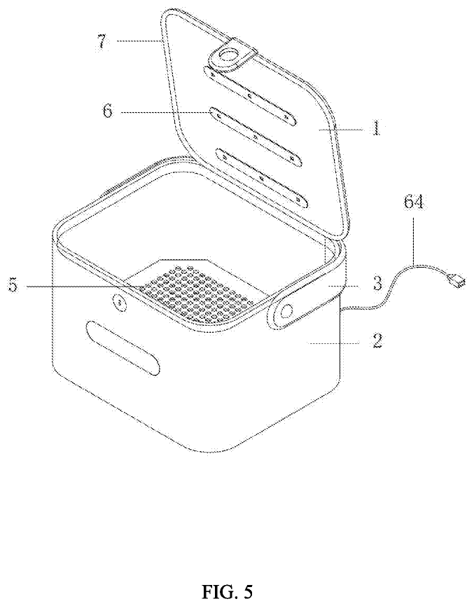

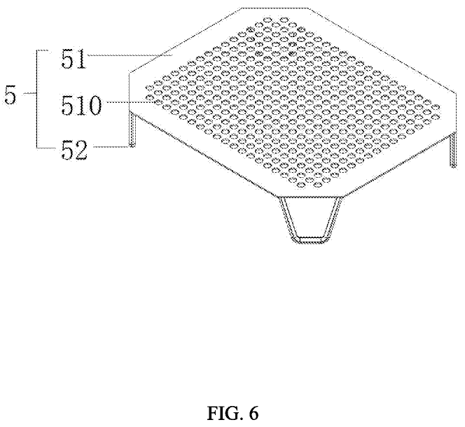

Referring to FIGS. 1-6, a UV disinfection package includes a main body 2, a bracket 5, a reflector 600, an aluminum film 103 and a UVC lamp group 6. The bracket 5 is fixedly installed in the main body 2. The UVC lamp group 6 is fixedly installed on the main body 2. The reflector 600 and the UVC lamp group 6 is fixedly connected. The aluminum film 103 covers the inner wall of the main body 2. The bracket 5 includes a light-transmitting hole 510, a storage layer 51 and a foot 52. The foot 52 is fixedly connected to the storage layer 51, and the light-transmitting hole 510 is provided on the storage layer 51.

In the present embodiment, the bracket 5 can be movably placed in the main body 2 to support the object to be disinfected. The foot 52 is used to support the storage layer 51. UV light can pass through the light-transmitting hole 510 to disinfect the bottom of the object. The UVC lamp group 6 is wrapped in the main body 2 by sewing. The UVC lamp can emit UV light with a UV wavelength of 240-280 nm, which can easily destroy the molecular structure of DNA (deoxyribonucleic acid) or RNA (ribonucleic acid) in the bacteria and viruses, resulting in the death of growth cells and/or regenerative cells, achieving the effect of sterilization and disinfection. The inner surface of the main body 2 is covered with a layer of aluminum film 103, which can play the role of reflection. At the same time, the reflector 600 is provided outside the UVC lamp group 6. The reflector 600 and the aluminum film 103 can reflect each other, so that the entire UV disinfection package reaches 360° of UV light irradiation without dead angle, and the object can be disinfected with UV in all directions.

Furthermore, the main body 2 is provided with an upper cover 1 and a slot hole 21. The upper cover 1 is provided with a pull buckle 11 which is snap connected with the slot hole 21. The UV disinfection package is provided with a magnetic strip 7, which includes a first magnetic strip 71, a second magnetic strip 72, a third magnetic strip 73 and a fourth magnetic strip 74. The first magnetic strip 71 is fixedly connected to the upper cover 1. The second magnetic strip 72 is fixedly connected to the main body 2. The third magnetic strip 73 is fixedly connected to the pull buckle 11. And the fourth magnetic strip 74 is fixedly installed at the edge of the slot hole 21.

In the present embodiment, the magnetic strip 7 is wrapped in the upper cover 1 and the main body 2 by sewing. The magnetic strip 7 has a mutual attraction function, which is more convenient for opening and closing the upper cover 1, and more convenient for the upper cover 1 to use. The first magnetic stripe 71 and the second magnetic stripe 72 magnetic attract each other, and the third magnetic stripe 73 and the fourth magnetic stripe 74 magnetic attract each other, so that the upper cover 1 and the main body 2 are connected by magnetic attraction, making the operation easier and more convenient.

Furthermore, the UVC light group 6 includes a UVC light stripe 61, a wire 63, a power cable 64, a connection terminal 62, a circuit control board 65, a switch button board 67, and a switch terminal 66. The UVC light stripe 61 is provided with a plurality of UVC lamp beads 610. A plurality of the UVC lamp stripes 61 are fixedly connected through the connection terminal 62, and the UVC lamp stripe 61 and the circuit control board 65 are fixedly connected through the wire 63. One end of the switch terminal 66 is fixedly connected to the circuit control board 65, and the other end of the switch terminal 66 is fixedly connected to the switch button board 67. The power cable 64 is fixedly connected to the circuit control board 65.

In the present embodiment, the UVC light stripe 61, the wire 63, the power cable 64, the connection terminal 62, the circuit control board 65, the switch button board 67 and the switch terminal 66 are electrically connected to form a control circuit, which controls the activation and deactivation of the UVC lamp group 6, thereby controlling the activation and deactivation of the UV disinfection package. After the object to be disinfected is placed in the disinfection package, a user can press the button on the switch button board 67 to perform disinfection for 3-5 minutes. In the process of disinfection, when the upper cover 1 is opened, the UVC light stripe 61 will be automatically extinguished, so that the UV light will not irradiate people's eyes and skin. The power cable 64 is a USB cable, which is more convenient for consumers to power on.

Furthermore, the main body 2 is composed of a surface layer 100, a paperboard layer 101 and an inner cloth 102. The surface layer 100, the paperboard layer 101, the inner cloth 102 and the aluminum film 103 are arranged and fixed in sequence.

In the present embodiment, the main body 2 is composed of the surface layer 100, the paperboard layer 101, the inner cloth 102 and the aluminum film 103, which are sewn together to achieve the effect of hard, thick and less deformable.

Furthermore, the UV disinfection package includes a window 4, which is provided on the main body 2.

In the present embodiment, the window 4 is made of a special transparent material that can completely block UV radiation. And through the window 4, it can be observed whether the UVC light stripe 61 is working.

Furthermore, the UV disinfection package includes a handle 3, which is fixedly connected to the main body 2.

In the present embodiment, the handle 3 enables the user to conveniently carry or move UV disinfection package, so that the UV disinfection package can be taken to different places to use.

Furthermore, the UV disinfection package includes a back pocket 22, which is fixedly connected to the main body 2.

In the present embodiment, the back pocket 22 provided in the UV disinfection package can receive the power cable 64 to make the entire UV disinfection package more beautiful.

The present disclosure provides a UV disinfection package which adopts a handbag-type design. This UV disinfection package is simple and stylish. It uses a magnetic attraction opening cover structure, making the opening and closing operations simple and the disinfection operation more convenient. A UVC lamp control circuit is adopted to control the UVC lamp to automatically disinfect for 3-5 minutes by pressing a button once. The lamp will automatically extinguish as a protection function after the cover is open, so as to avoid hurting people's eyes and skin due to the UV radiation. With multiple lamp beads, an aluminum film and a reflector reflecting the UV light together, the entire disinfection package reaches 360° of UV light irradiation without dead angle. A bracket is used to support the object to be disinfected. The bracket is provided with a light-transmitting hole, which is convenient for the reflector and the aluminum film to reflect each other, so as to reach 360° of UV light irradiation without dead angle. The UV light with UV wavelength of 240-280 nm can destroy the molecular structure of DNA or RNA in the bacteria and viruses, resulting in the death of cells. Multi-layer sewing is used to make the package tougher and less deformable, and the disinfection is safe and thorough.

Although the features and elements of the present disclosure are described as embodiments in particular combinations, each feature or element can be used alone or in other various combinations within the principles of the present disclosure to the full extent indicated by the broad general meaning of the terms in which the appended claims are expressed.

What is claimed is:

1. A UV disinfection package comprising:
a main body;
a bracket fixedly installed in the main body, wherein the bracket comprises a light-transmitting hole, a storage layer and a foot, the foot being fixedly connected to the storage layer, and the light-transmitting hole being provided on the storage layer;
a UVC lamp group fixedly installed on the main body;
a reflector fixedly connected to the UVC lamp group; and
an aluminum film covering the inner wall of the main body.

2. The UV disinfection package of claim 1, wherein the main body is provided with an upper cover and a slot hole, the upper cover being provided with a pull buckle which is snap connected with the slot hole.

3. The UV disinfection package of claim 2 being provided with a magnetic strip, wherein the magnetic strip comprises a first magnetic strip, a second magnetic strip, a third magnetic strip and a fourth magnetic strip, the first magnetic strip being fixedly connected to the upper cover, the second magnetic strip being fixedly connected to the main body, the third magnetic strip being fixedly connected to the pull buckle, and the fourth magnetic strip being fixedly installed at the edge of the slot hole.

4. The UV disinfection package of claim 1, wherein the UVC lamp group comprises a UVC lamp stripe, a wire, a power cable, a connection terminal, a circuit control board, a switch button board and a switch terminal, the UVC lamp stripe being provided with a plurality of UVC lamp beads, a plurality of the UVC lamp stripes being fixedly connected through the connection terminal, the UVC lamp stripe and the circuit control board being fixedly connected through the wire, one end of the switch terminal being fixedly connected to the circuit control board, the other end of the switch terminal being fixedly connected to the switch button board, and the power cable being fixedly connected with the circuit control board.

5. The UV disinfection package of claim 1, wherein the main body is composed of a surface layer, a paperboard layer and an inner cloth, wherein the surface layer, the paperboard layer, the inner cloth and the aluminum film are arranged and fixed in sequence.

6. The UV disinfection package of claim 1, further comprising a window which is provided on the main body.

7. The UV disinfection package of claim 1, further comprising a handle which is fixedly connected to the main body.

8. The UV disinfection package of claim 1, further comprising a back pocket which is fixedly connected to the main body.
